(19) United States
(12) Patent Application Publication
(10) Pub. No.: US 2009/0069843 A1
(43) Pub. Date: Mar. 12, 2009

(54) FISTULA PLUGS INCLUDING A HYDRATION RESISTANT COMPONENT

(76) Inventor: Charles W. Agnew, West Lafayette, IN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 12/181,660

(22) Filed: Jul. 29, 2008

Related U.S. Application Data
(60) Provisional application No. 60/971,091, filed on Sep. 10, 2007.

(51) Int. Cl. A61B 17/03

(52) U.S. Cl. 606/213

(57) ABSTRACT

Described, in certain aspects, are devices and methods for treating fistulae. In one embodiment, a fistula plug includes a hydratable component and hydration resistant component incorporated on or in the hydratable component. Illustratively, an inventive plug can include a first component and a second component, wherein the first component is hydratable, and the second component is less receptive to hydration than the first component (or is essentially non-hydratable). Either of these components may be formed with one or more of a variety of biocompatible materials including some that are naturally derived and some that are non-naturally derived. In one embodiment, the first component and the second component, while dissimilar in their receptivity to hydration, are both comprised of a remodelable, angiogenic material, for example, a remodelable extracellular matrix material such as submucosa.

### REFERENCE TO RELATED APPLICATION

The present application claims the benefit of U.S. Provisional Patent Application Ser. No. 60/971,091 filed Sep. 10, 2007 entitled FISTULA PLUGS INCLUDING A HYDRATION RESISTANT COMPONENT which is hereby incorporated by reference in its entirety.

### BACKGROUND

The present invention relates generally to medical devices and in particular aspects to devices and methods for plugging fistulae and other passageways in the body.

As further background, there exist a variety of passages and other open spaces in the body which can be plugged or otherwise filled to provide benefit to the patient. For example, it may be desirable to occlude a lumen or other open space in the vasculature (e.g., a blood vessel such as a vein or artery). In some instances, a device is deployed within the venous system, e.g., within the greater and/or lesser saphenous vein, to treat complications, such as a varicose vein conditions.

As well, it may be desirable to plug or otherwise fill a fistula. A variety of fistulae can occur in humans. These fistulae can occur for a variety of reasons, such as but not limited to, as a congenital defect, as a result of inflammatory bowel disease, such as Crohn's disease, irradiation, trauma, such as childbirth, or as a side effect from a surgical procedure. Further, several different types of fistulae can occur, for example, urethro-vaginal fistulae, vesico-vaginal fistulae, tracheo-esophageal fistulae, gastro-cutaneous fistulae, and any number of anorectal fistulae, such as recto-vaginal fistula, recto-vesical fistulae, recto-urethral fistulae, or recto-prostatic fistulae.

The path which fistulae take, and their complexity, can vary. A fistula may take a take a “straight line” path from a primary opening to a secondary opening, known as a simple fistula. Alternatively, a fistula may comprise multiple tracts ramifying from a primary opening and have multiple secondary openings. This is known as a complex fistula.

Anorectal fistulae can result from infection in the anal glands, which are located around the circumference of the distal anal canal that forms the anatomic landmark known as the dentate line. Approximately 20-40 such glands are found in humans. Infection in an anal gland can result in an abscess. This abscess then can track through soft tissues (e.g., through or around the sphincter muscles) into the perianal skin, where it drains either spontaneously or surgically. The resulting void through soft tissue is known as a fistula. The internal or inner opening of the fistula, usually located at or near the dentate line, is known as the primary opening. Any external or outer openings, which are usually located in the perianal skin, are known as secondary openings.

One technique for treating a perianal fistula is to make an incision adjacent the anus until the incision contacts the fistula and then excise the fistula from the anal tissue. This surgical procedure tends to sever the fibers of the anal sphincter, and may cause incontinence. Other surgical treatment of fistulae involve passing a fistula probe through the tract of the fistula in a blind manner, using primarily only tactile sensation and experience to guide to probe. Having passed the probe through the fistula tract, the overlying tissue is surgically divided. This is known as a fistulotomy. Since a variable amount of sphincter muscle is divided during the procedure, fistulotomy also may result in impaired sphincter control, and even frank incontinence.

A gastrointestinal fistula is an abnormal passage that leaks contents of the stomach or the intestine (small or large bowel) to other organs, usually other parts of the intestine or the skin. For example, gastrojejunocolic fistulae include both enterocutaneous fistulae (those occurring between the skin surface and the intestine, namely the duodenum, the jejunum, and the ileum) and gastric fistulae (those occurring between the stomach and skin surface). Another type of fistula occurring in the gastrointestinal tract is an enteroenteral fistula, which refers to a fistula occurring between two parts of the intestine. Gastrointestinal fistulae can result in malnutrition and dehydration depending on their location in the gastrointestinal tract. They can also be a source of skin problems and infection. The majority of these types of fistulae are the result of surgery (e.g., bowel surgery), although sometimes they can develop spontaneously or from trauma, especially penetrating traumas such as stab wounds or gunshot wounds. Inflammatory processes, such as infection or inflammatory bowel disease (Crohn's disease), may also cause gastrointestinal fistulae. In fact, Crohn's disease is the most common primary bowel disease leading to enterocutaneous fistulae, and surgical treatment may be difficult because additional enterocutaneous fistulae develop in many of these patients postoperatively.

Treatment options for gastrointestinal fistulae vary. Depending on the clinical situation, patients may require IV nutrition and a period of time without food to allow the fistula time to close on its own. Indeed, nonsurgical therapy may allow spontaneous closure of the fistula, although this can be expected less than 30% of the time according to one estimate. A variable amount of time to allow spontaneous closure of fistulae has been recommended, ranging from 30 days to 6 to 8 weeks. During this preoperative preparation, external control of the fistula drainage prevents skin disruption and provides guidelines for fluid and electrolyte replacement. In some cases, surgery is necessary to remove the segment of intestine involved in a non-healing fistula.

When surgery is deemed necessary, one operation for fistula closure is resection of the fistula-bearing segment and primary end-to-end anastamosis. The anastomosis may be reinforced by greater momentum or a serosal patch from adjacent small bowel. Still other methods for treating fistulae involve injecting sclerosant or sealant (e.g., collagen or fibrin glue) into the tract of the fistula to block the fistula. Closure of a fistula using a sealant is typically performed as a two-stage procedure, including a first-stage seton placement and injection of the fibrin glue several weeks later. This allows residual infection to resolve and to allow the fistula tract to “mature” prior to injecting a sealant. If sealant or sclerosant were injected as a one-stage procedure, into an “unprepared” or infected fistula, this may cause a flare-up of the infection and even further abscess formation.

There remain needs for improved and/or alternative devices and methods for plugging passageways and other open spaces in the body. The present invention is addressed to those needs.

### SUMMARY

The present invention provides, in certain aspects, unique devices for plugging passageways and other open spaces in the body. In some forms, devices of this sort include material in a core region of the device that is more resistant to hydration than material in one or more other regions of the device (e.g., non-core regions). Illustratively, one such device is a fistula plug for delivery into a fistula tract, wherein the fistula plug includes a plug body and a core material received in the plug body. The plug body is comprised of a dried collagen-containing material, and the core material is less receptive to hydration than the plug body. The core material may or may not contain collagen. Thus, although not necessary to broader aspects of the invention, in some embodiments, the core material and the plug body, while dissimilar in their receptivity to hydration, will be comprised of one or more of the same materials. In some preferred aspects, the plug body and/or the core material include a remodelable, angiogenic material, for example, a remodelable extracellular matrix material such as submucosa. The fistula plug, as well as any of its components, can be shaped and configured in a variety of manners. The core material may or may not be removable from the plug body. In one aspect, the plug body provides a designated opening (e.g., a lumen or other passage) in which the core material is removably positioned.

In another embodiment, the invention provides a fistula plug that includes a plug body and a hydration resistant material component. The plug body is comprised of a hydratable material, and the hydration resistant material component is incorporated on or in the plug body. The hydratable material can be a variety of materials, and in some embodiments, will include a naturally-derived material, a non-naturally-derived material, or both. Illustratively, the hydratable material may include a collagen-containing material such as a collagenous extracellular matrix material. The hydration resistant material component may be comprised of one or more of a variety of materials as well, and can exhibit any suitable size, shape and configuration for incorporation on or in the plug body. Illustratively, the hydration resistant material component may include a sheet-form material and/or a non-sheet-form material. In one embodiment, the hydratable material is comprised of a porous material having a plurality of interconnected spaces therein, and the hydration resistant material component includes material residing in the interconnected spaces.

One aspect of the present invention provides a method of treating a fistula having at least a primary fistula opening, a secondary fistula opening, and a fistula tract extending therebetween. This method includes delivering into the fistula tract a fistula plug such as that described above. Delivery of this sort can be accomplished in a variety of manners including some that involve pushing and/or pulling the fistula plug in the fistula tract, e.g., through the primary fistula opening and toward the secondary fistula opening, or vice versa. In some embodiments, the fistula plug is delivered into the fistula tract in a delivery device lumen.

Another aspect of the invention provides a fistula plug including a plug body comprised of a rolled sheet-form material. The plug body includes a collagen-containing material layer and a hydration resistant material layer. While not necessary to broader aspects of the invention, in certain embodiments, the collagen-containing material layer surrounds at least a portion of the hydration resistant material layer. The plug body can exhibit a variety of shapes and sizes, and in some forms, will include a generally cylindrical portion and/or a generally conical portion.

A further embodiment of the invention provides a fistula plug that includes a plug body and a hydration resistant coating material. The plug body is comprised of a collagen-containing material, and the hydration resistant coating material coats a surface of the plug body. The coating material may, in some aspects, coat an interior surface of the plug body, an exterior surface of the plug body, or both. In one form, the collagen-containing material is comprised of a material layer, and the coating material coats a surface of this layer.

Yet another embodiment of the present invention provides a fistula plug that includes an articulating plug component comprised of two or more elongate plug body segments hingedly connected to one another in succession. In one form, the plug further comprises a covering material positioned around the two or more elongate plug body segments, for example, a sheet-form material wrapped around the segments. The two or more elongate plug body segments can each exhibit a variety of shapes and sizes, and the segments may be hingedly connected to one another in a variety of manners including but not limited to with suture material and other one or multiple-part devices and materials. Suitable plug body segments, in some embodiments, are comprised of material that is rolled, folded, braided, etc.

In another aspect, the invention provides a method of plugging a passageway in the body. This method comprises delivering into the body passageway a plugging device comprised of a hydrated remodelable angiogenic material, wherein the hydrate in the material is frozen. In some forms, the remodelable angiogenic material comprises an extracellular matrix material such as but not limited to porcine small intestine submucosa.

Other objects, embodiments, forms, features, advantages, aspects, and benefits of the present invention shall become apparent from the detailed description and drawings included herein.

### BRIEF DESCRIPTION OF THE DRAWINGS

FIG. 1 is a perspective view of fistula plug according to one embodiment of the present invention.

FIG. 2 is a partial, perspective view of another fistula plug of the invention.

FIG. 3 is a perspective view of a fistula plug according to another embodiment of the invention.

FIG. 4 is a partial, perspective view of another fistula plug of the invention.

FIG. 5 shows another fistula plug according to the present invention.

FIG. 6 shows a fistula plug in accordance with another embodiment of the present invention.

FIG. 7 shows another fistula plug of the invention.

### DETAILED DESCRIPTION

While the present invention may be embodied in many different forms, for the purpose of promoting an understanding of the principles of the present invention, reference will now be made to the embodiments illustrated in the drawings, and specific language will be used to describe the same. It will nevertheless be understood that no limitation of the scope of the invention is thereby intended. Any alterations and further modifications in the described embodiments and any further applications of the principles of the present invention as described herein are contemplated as would normally occur to one skilled in the art to which the invention relates.

As disclosed above, in certain aspects, the present invention provides unique devices and methods for treating fistulae. These devices, in some embodiments, include a hydration resistant component. A component of this sort can exist in various forms in an inventive device. In some forms, a hydration resistant component provides one or more regions or other parts of a device that are effective to enhance the hydration resistance characteristics of the device as a whole. Such regions can include material that has been physically, chemically, biologically and/or otherwise treated to alter its resistance to hydration. Additionally or alternatively, such regions or parts can be provided, for example, by one or more objects (e.g., material layers, particles, formed constructs, etc.) that are connected to, embedded within or otherwise incorporated on or in a device.

In one aspect, the invention provides a fistula plug comprised of a first component and a second component, wherein the first component is hydratable, and the second component is less receptive to hydration than the first component (or is essentially non-hydratable). Either of these components may be formed with one or more of a variety of biocompatible materials including some that are naturally derived and some that are non-naturally derived. In a preferred embodiment, the first component and the second component, while dissimilar in their receptivity to hydration, are both comprised of a collagen-containing material, for example, a remodelable, angiogenic extracellular matrix material such as submucosa.

The invention further provides methods for preparing and using these and other inventive devices, as well as medical products that include such devices enclosed within sterile packaging. Some aspects of the invention involve the treatment of fistulae having at least a primary fistula opening, a secondary fistula opening and a fistula tract extending therebetween. Illustratively, a fistula treatment method can include delivering into a fistula tract a device such as any of those described herein. In instances where the incorporation of a hydration resistant component increases the column strength of an elongate device, this increase may be effective to enhance one or more delivery characteristics of the device. Such devices, potentially also exhibiting some degree of lateral flexibility, may be particularly useful in instances where the device is to be delivered into and through a long, wet fistula tract. These devices may be pushed and/or pulled in the tract during placement.

In some embodiments, one or more hydration resistant material layers provide a hydration resistant component. When present in a device, a material layer of this sort can be incorporated into the device in a variety of manners. Although not necessary to broader aspects of the invention, in some forms, such a layer will be wholly or partially embedded within or otherwise incorporated on or in other parts of a device, for example, as a covering to a hydratable plug body formed with layered and/or non-layered material. When a device includes layers having differing properties with regard to hydration resistance, any material layer present in the device may be arranged in any suitable fashion including some that involve folding, rolling and/or otherwise overlaying portions of material. In one aspect, a hydration resistant material layer provides an interior component of a plug device.

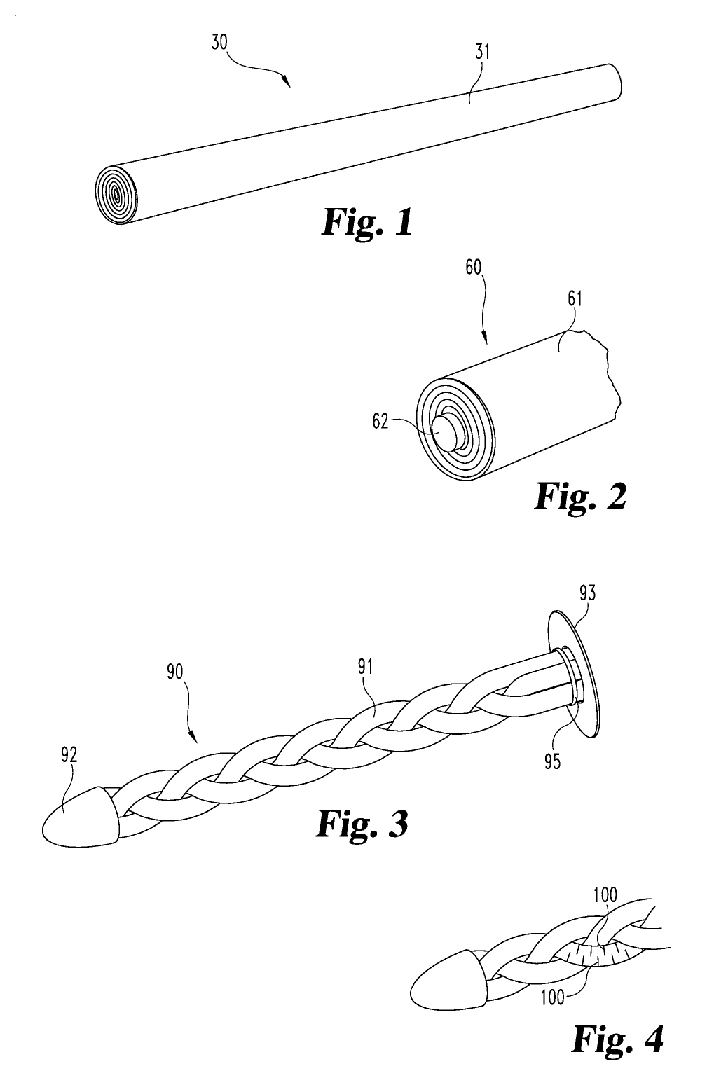

With reference now to FIG. 1, shown is a fistula plug 30 including a plug body 31. Plug body 31 is comprised of a rolled sheet-form material exhibiting a gently tapered, nearly cylindrical shape. The sheet-form material includes a first material layer in an overlapping relationship with a second material layer, wherein the first material layer is hydratable, and the second material layer is less receptive to hydration than the first material layer. In this specific illustrative embodiment, the 2-layer material is rolled such that the second, relatively less hydratable material layer provides a substantial portion of the outer surface of plug body 31. Providing a sheet having at least two layers can be accomplished in a variety of manners. In some aspects, two separate and distinct material layers are joined together to form a multilayered construct. Additionally or alternatively, a material layer can be formed onto another material layer, for example, as a flowable material sprayed onto or otherwise applied to an existing material layer.

Plug bodies useful in the invention such as plug body 31 can be shaped and configured in a variety of manners. In some forms, a device includes an elongate graft body having either a constant or varying cross-sectional area along its length, or portions thereof. Illustratively, all or part of a graft body can exhibit a generally cylindrical shape, a conical shape, and other suitable shapes including some having tapered and/or non-tapered longitudinal portions. As well, a cross section of a particular graft body portion can exhibit a variety shapes including some that have rectilinear and/or curvilinear portions. Thus, a graft body can include a portion having a generally circular or non-circular (e.g., elliptical, square, star-shaped, hexagonal, etc.) cross section.

In embodiments where an inventive device is configured for positioning in a fistula tract, such a device will generally be configured to extend through the tract (or a segment thereof), and in some cases, will be sufficient to fill or substantially fill at least a segment of the tract. In certain embodiments, a device will have a length of at least about 0.20 cm, and in many instances at least about 1 cm to about 20 cm (approximately 1 to 8 inches) for positioning in a fistula tract. In some cases, a device will have a length of from about 2 cm to about 5 cm, or alternatively, from about 2 inches to about 4 inches. Additionally, a device useful in the invention, or any portion thereof, can have a diameter, which may or may not be constant along its length, from about 0.1 mm to about 25 mm, or more typically from about 5 mm to about 15 mm. In certain forms, a generally conical device is tapered along its length so that one end of the device has a diameter of about 5 mm to about 15 mm, while the opposite end of the device has a diameter of about 0.5 mm to about 5 mm. Such a taper may or may not be continuous along the length of the device.

In certain aspects, formation of a rolled plug body such as plug body 31 involves wrapping one or more material layers around a mandrel or otherwise applying material to a suitable supporting device such as a mold or form. Illustratively, a hydratable, first material layer can be overlapped with (and potentially attached to) a non-hydratable, second material layer (or a relatively less hydratable material layer), and then the 2-layer construct can be wrapped around a mandrel one or more times as part of forming a plug. Once wrapped fully around, the outer edge of the 2-layer construct can then be fixed to an underlying wrapped portion. Additionally or alternatively, a thin layer of adhesive can be applied to each successive underlying layer as the construct is wrapped around the mandrel so that a substantial portion of the rolled layers are adhered to one another. Any of these techniques may additionally involve compression and drying steps.

Alternatively, formation of a plug can include wrapping a hydratable, first material layer around a mandrel one or more times, and then wrapping a relatively less hydratable, second material layer around the mandrel (atop the first material layer) one or more times, or vice versa. Material layers of the same or different dimensions (including thickness) can be combined to form an inventive plug. In certain aspects, when a first material layer is wrapped around a second material layer, the first material layer wholly or partially overlaps the second material layer.

In this regard, some of the plug bodies useful in the invention can be formed by folding or rolling, or otherwise overlaying one or more portions of a biocompatible material, such as a biocompatible sheet material. In some aspects, the overlaid biocompatible sheet material is then compressed and dried or otherwise bonded into a volumetric shape such that a substantially unitary construct is formed. In some forms, a plug body is constructed by randomly or regularly packing one or more pieces of single or multilayer ECM sheet material within a mold and thereafter processing the packed material. Plug bodies useful in the invention can be prepared, for example, as described in International Patent Application Serial No. PCT/US2006/16748, filed Apr. 29, 2006, and entitled “VOLUMETRIC GRAFTS FOR TREATMENT OF FISTULAE AND RELATED METHODS AND SYSTEMS” (Cook Biotech Incorporated), which is hereby incorporated by reference in its entirety.

Additionally, one or more hydration resistant material layers may be incorporated on or in a hydratable plug portion that includes material not in layer form. Such “non-layered” material can be formed in any suitable manner including but not limited to by extrusion, using a mold or form, construction around a mandrel, and/or combinations or variations thereof. In some embodiments, such a portion is formed with a reconstituted or otherwise reassembled ECM material. When combined with such a portion, any hydration resistant material layer present in a plug of this sort may be arranged in any suitable fashion in the plug including arrangements that involve folding, rolling and/or otherwise overlaying material. Illustratively, a hydratable component formed with a non sheet-form material can be partially, and in some embodiments wholly, surrounded by a sheet-form hydration resistant material.

When an inventive device includes two components dissimilar in their resistance to hydration, these two components may or may not be formed with one or more of the same materials. In certain embodiments, an inventive plug includes a first component and a second component comprised of the same material, yet the first component is altered to make it more resistant to hydration than the second component. Such an alteration can involve adding a substance to a component, subtracting a substance from a component and/or otherwise manipulating one or more physical, chemical, biological or other properties of a component. In some instances a substance is added to a material as a coating to make it more hydration resistant, for example, by spray coating, dip coating, etc. When a component comprises a porous material having a plurality of interconnected spaces therein, a hydration resistance altering substance can be positioned in these spaces, for example, by soaking the porous material in the substance. A variety of other ways to alter the hydration resistance of a material will be recognized by those skilled in the art, and therefore, are encompassed by the present invention. These include but are not limited to increasing the density of a porous material, and then stabilizing the material in this higher density state. Additionally or alternatively, a variety of hydrophobic materials including various hydrophobic polymers, waxes and oils can be incorporated into inventive devices.

Turning now to a more detailed discussion of materials useful in forming devices of the invention, these materials should generally be biocompatible, and in advantageous embodiments of the devices, are comprised of a remodelable material. Particular advantage can be provided by devices including a remodelable collagenous material. Such remodelable collagenous materials, whether reconstituted or naturally-derived, can be provided, for example, by collagenous materials isolated from a warm-blooded vertebrate, and especially a mammal. Such isolated collagenous material can be processed so as to have remodelable, angiogenic properties and promote cellular invasion and ingrowth. Remodelable materials may be used in this context to promote cellular growth on, around, and/or within tissue in which a device of the invention is implanted, e.g., around tissue defining a fistula tract, an opening to a fistula, or another space in the body.

Suitable remodelable materials can be provided by collagenous extracellular matrix (ECM) materials possessing biotropic properties. For example, suitable collagenous materials include ECM materials such as those comprising submucosa, renal capsule membrane, dermal collagen, dura mater, pericardium, fascia lata, serosa, peritoneum or basement membrane layers, including liver basement membrane. Suitable submucosa materials for these purposes include, for instance, intestinal submucosa including small intestinal submucosa, stomach submucosa, urinary bladder submucosa, and uterine submucosa. Collagenous matrices comprising submucosa (potentially along with other associated tissues) useful in the present invention can be obtained by harvesting such tissue sources and delaminating the submucosa-containing matrix from smooth muscle layers, mucosal layers, and/or other layers occurring in the tissue source. For additional information as to some of the materials useful in the present invention, and their isolation and treatment, reference can be made, for example, to U.S. Pat. Nos. 4,902,508, 5,554,389, 5,993,844, 6,206,931, and 6,099,567.

Submucosa-containing or other ECM tissue used in the invention is preferably highly purified, for example, as described in U.S. Pat. No. 6,206,931 to Cook et al. Thus, preferred ECM material will exhibit an endotoxin level of less than about 12 endotoxin units (EU) per gram, more preferably less than about 5 EU per gram, and most preferably less than about 1 EU per gram. As additional preferences, the submucosa or other ECM material may have a bioburden of less than about 1 colony forming units (CFU) per gram, more preferably less than about 0.5 CFU per gram. Fungus levels are desirably similarly low, for example less than about 1 CFU per gram, more preferably less than about 0.5 CFU per gram. Nucleic acid levels are preferably less than about 5 μg/mg, more preferably less than about 2 μg/mg, and virus levels are preferably less than about 50 plaque forming units (PFU) per gram, more preferably less than about 5 PFU per gram. These and additional properties of submucosa or other ECM tissue taught in U.S. Pat. No. 6,206,931 may be characteristic of any ECM tissue used in the present invention.

A typical layer thickness for an as-isolated submucosa or other ECM tissue layer used in the invention ranges from about 50 to about 250 microns when fully hydrated, more typically from about 50 to about 200 microns when fully hydrated, although isolated layers having other thicknesses may also be obtained and used. These layer thicknesses may vary with the type and age of the animal used as the tissue source. As well, these layer thicknesses may vary with the source of the tissue obtained from the animal source.

Suitable bioactive agents may include one or more bioactive agents native to the source of the ECM tissue material. For example, a submucosa or other remodelable ECM tissue material may retain one or more growth factors such as but not limited to basic fibroblast growth factor (FGF-2), transforming growth factor beta (TGF-beta), epidermal growth factor (EGF), cartilage derived growth factor (CDGF), and/or platelet derived growth factor (PDGF). As well, submucosa or other ECM materials when used in the invention may retain other native bioactive agents such as but not limited to proteins, glycoproteins, proteoglycans, and glycosaminoglycans. For example, ECM materials may include heparin, heparin sulfate, hyaluronic acid, fibronectin, cytokines, and the like. Thus, generally speaking, a submucosa or other ECM material may retain one or more bioactive components that induce, directly or indirectly, a cellular response such as a change in cell morphology, proliferation, growth, protein or gene expression.

Submucosa or other ECM materials of the present invention can be derived from any suitable organ or other tissue source, usually sources containing connective tissues. The ECM materials processed for use in the invention will typically include abundant collagen, most commonly being constituted at least about 80% by weight collagen on a dry weight basis. Such naturally-derived ECM materials will for the most part include collagen fibers that are non-randomly oriented, for instance occurring as generally uniaxial or multi-axial but regularly oriented fibers. When processed to retain native bioactive factors, the ECM material can retain these factors interspersed as solids between, upon and/or within the collagen fibers. Particularly desirable naturally-derived ECM materials for use in the invention will include significant amounts of such interspersed, non-collagenous solids that are readily ascertainable under light microscopic examination with appropriate staining. Such non-collagenous solids can constitute a significant percentage of the dry weight of the ECM material in certain inventive embodiments, for example at least about 1%, at least about 3%, and at least about 5% by weight in various embodiments of the invention.

The submucosa or other ECM material used in the present invention may also exhibit an angiogenic character and thus be effective to induce angiogenesis in a host engrafted with the material. In this regard, angiogenesis is the process through which the body makes new blood vessels to generate increased blood supply to tissues. Thus, angiogenic materials, when contacted with host tissues, promote or encourage the formation of new blood vessels into the materials. Methods for measuring in vivo angiogenesis in response to biomaterial implantation have recently been developed. For example, one such method uses a subcutaneous implant model to determine the angiogenic character of a material. See, C. Heeschen et al., Nature Medicine 7 (2001), No. 7, 833-839. When combined with a fluorescence microangiography technique, this model can provide both quantitative and qualitative measures of angiogenesis into biomaterials. C. Johnson et al., Circulation Research 94 (2004), No. 2, 262-268.

Further, in addition or as an alternative to the inclusion of such native bioactive components, non-native bioactive components such as those synthetically produced by recombinant technology or other methods (e.g., genetic material such as DNA), may be incorporated into an ECM material. These non-native bioactive components may be naturally-derived or recombinantly produced proteins that correspond to those natively occurring in an ECM tissue, but perhaps of a different species. These non-native bioactive components may also be drug substances. Illustrative drug substances that may be added to materials include, for example, anti-clotting agents, e.g. heparin, antibiotics, anti-inflammatory agents, thrombus-promoting substances such as blood clotting factors, e.g., thrombin, fibrinogen, and the like, and anti-proliferative agents, e.g. taxol derivatives such as paclitaxel. Such non-native bioactive components can be incorporated into and/or onto ECM material in any suitable manner, for example, by surface treatment (e.g., spraying) and/or impregnation (e.g., soaking), just to name a few. Also, these substances may be applied to the ECM material in a premanufacturing step, immediately prior to the procedure (e.g., by soaking the material in a solution containing a suitable antibiotic such as cefazolin), or during or after engraftment of the material in the patient.

Devices of the invention can include xenograft material (i.e., cross-species material, such as tissue material from a non-human donor to a human recipient), allograft material (i.e., interspecies material, with tissue material from a donor of the same species as the recipient), and/or autograft material (i.e., where the donor and the recipient are the same individual). Further, any exogenous bioactive substances incorporated into an ECM material may be from the same species of animal from which the ECM material was derived (e.g. autologous or allogenic relative to the ECM material) or may be from a different species from the ECM material source (xenogenic relative to the ECM material). In certain embodiments, ECM material will be xenogenic relative to the patient receiving the graft, and any added exogenous material(s) will be from the same species (e.g. autologous or allogenic) as the patient receiving the graft. Illustratively, human patients may be treated with xenogenic ECM materials (e.g. porcine-, bovine- or ovine-derived) that have been modified with exogenous human material(s) as described herein, those exogenous materials being naturally derived and/or recombinantly produced.

ECM materials used in the invention may be essentially free of additional, non-native crosslinking, or may contain additional crosslinking. Such additional crosslinking may be achieved by photo-crosslinking techniques, by chemical crosslinkers, or by protein crosslinking induced by dehydration or other means. However, because certain crosslinking techniques, certain crosslinking agents, and/or certain degrees of crosslinking can destroy the remodelable properties of a remodelable material, where preservation of remodelable properties is desired, any crosslinking of the remodelable ECM material can be performed to an extent or in a fashion that allows the material to retain at least a portion of its remodelable properties. Chemical crosslinkers that may be used include for example aldehydes such as glutaraldehydes, diimides such as carbodiimides, e.g., 1-ethyl-3-(3-dimethylaminopropyl)carbodiimide hydrochloride, ribose or other sugars, acyl-azide, sulfo-N-hydroxysuccinamide, or polyepoxide compounds, including for example polyglycidyl ethers such as ethyleneglycol diglycidyl ether, available under the trade name DENACOL EX810 from Nagese Chemical Co., Osaka, Japan, and glycerol polyglycerol ether available under the trade name DENACOL EX 313 also from Nagese Chemical Co. Typically, when used, polyglycerol ethers or other polyepoxide compounds will have from 2 to about 10 epoxide groups per molecule.

Turning now to a discussion of drying techniques that can be useful in certain embodiments of the invention, drying by evaporation, or air drying, generally comprises drying a partially or completely hydrated remodelable material by allowing the hydrant to evaporate from the material. Evaporative cooling can be enhanced in a number of ways, such as by placing the material in a vacuum, by blowing air over the material, by increasing the temperature of the material, by applying a blotting material during evaporation, or by any other suitable means or any suitable combination thereof. The amount of void space or open matrix structure within an ECM material that has been dried by evaporation is typically more diminished than, for example, an ECM material dried by lyophilization as described below.

A suitable lyophilization process can include providing an ECM material that contains a sufficient amount of hydrant such that the voids in the material matrix are filled with the hydrant. The hydrant can comprise any suitable hydrant known in the art, such as purified water or sterile saline, or any suitable combination thereof. Illustratively, the hydrated material can be placed in a freezer until the material and hydrant are substantially in a frozen or solid state. Thereafter, the frozen material and hydrant can be placed in a vacuum chamber and a vacuum initiated. Once at a sufficient vacuum, as is known in the art, the frozen hydrant will sublime from the material, thereby resulting in a dry remodelable material.

In alternative embodiments, a hydrated ECM material can be lyophilized without a separately performed pre-freezing step. In these embodiments, a strong vacuum can be applied to the hydrated material to result in rapid evaporative cooling which freezes the hydrant within the ECM material. Thereafter, the frozen hydrant can sublime from the material thereby drying the ECM material. Desirably, an ECM material that is dried via lyophilization maintains a substantial amount of the void space, or open matrix structure, that is characteristic of the harvested ECM material.

Drying by vacuum pressing generally comprises compressing a fully or partially hydrated remodelable material while the material is subject to a vacuum. One suitable method of vacuum pressing comprises placing a remodelable material in a vacuum chamber having collapsible walls. As the vacuum is established, the walls collapse onto and compress the material until it is dry. Similar to evaporative drying, when a remodelable material is dried in a vacuum press, more of the material's open matrix structure is diminished or reduced than if the material was dried by lyophilization.

In certain aspects, the invention provides devices, assemblies, etc. that include a multilaminate material. Such multilaminate materials can include a plurality of ECM material layers bonded together, a plurality of non-ECM materials bonded together, or a combination of one or more ECM material layers and one or more non-ECM material layers bonded together. To form a multilaminate ECM material, for example, two or more ECM segments are stacked, or one ECM segment is folded over itself at least one time, and then the layers are fused or bonded together using a bonding technique, such as chemical cross-linking or vacuum pressing during dehydrating conditions. An adhesive, glue or other bonding agent may also be used in achieving a bond between material layers. Suitable bonding agents may include, for example, collagen gels or pastes, gelatin, or other agents including reactive monomers or polymers, for example cyanoacrylate adhesives. As well, bonding can be achieved or facilitated between ECM material layers using chemical cross-linking agents such as those described above. A combination of one or more of these with dehydration-induced bonding may also be used to bond ECM material layers to one another.

A variety of dehydration-induced bonding methods can be used to fuse together portions of an ECM material. In one preferred embodiment, multiple layers of ECM material are compressed under dehydrating conditions. In this context, the term “dehydrating conditions” is defined to include any mechanical or environmental condition which promotes or induces the removal of water from the ECM material. To promote dehydration of the compressed ECM material, at least one of the two surfaces compressing the matrix structure can be water permeable. Dehydration of the ECM material can optionally be further enhanced by applying blotting material, heating the matrix structure or blowing air, or other inert gas, across the exterior of the compressed surfaces. One particularly useful method of dehydration bonding ECM materials is lyophilization.

Another method of dehydration bonding comprises pulling a vacuum on the assembly while simultaneously employing the vacuum to press the assembly together. Again, this method is known as vacuum pressing. During vacuum pressing, dehydration of the ECM materials in forced contact with one another effectively bonds the materials to one another, even in the absence of other agents for achieving a bond, although such agents can be used while also taking advantage at least in part of the dehydration-induced bonding. With sufficient compression and dehydration, the ECM materials can be caused to form a generally unitary ECM structure.

It is advantageous in some aspects of the invention to perform drying and other operations under relatively mild temperature exposure conditions that minimize deleterious effects upon any ECM materials being used, for example native collagen structures and potentially bioactive substances present. Thus, drying operations conducted with no or substantially no duration of exposure to temperatures above human body temperature or slightly higher, say, no higher than about 38° C., will preferably be used in some forms of the present invention. These include, for example, vacuum pressing operations at less than about 38° C., forced air drying at less than about 38° C., or either of these processes with no active heating—at about room temperature (about 25° C.) or with cooling. Relatively low temperature conditions also, of course, include lyophilization conditions.

Methods for forming graft bodies useful in the invention can involve manipulating a material within a mold or form. It should be noted that this material may or may not be hydrated when placed in, on, around, etc. the mold or form. In some methods, a substantially dry ECM material (e.g., a powder or sheet material) can be placed in a mold and then suitably hydrated for further processing. In other methods, a hydrated starting material is placed in and/or on a mold or forming structure for further processing. For example, one or more hydrated sheets of ECM material can be applied to a form, e.g., wrapped at least partially around a mandrel so that portions of the sheet(s) overlap. Then, the one or more sheets can be dried, and in some embodiments, dried while under compression, to form a unitary graft construct.

In some modes of operation, a hydrated graft material is provided within a single- or multiple-part mold having a plurality of apertures or holes extending through a wall of the mold, thereby providing access to the mold interior from an external location. These apertures can serve to enhance drying of a hydrated material during a processing step and in processes exerting vacuum pressure at these apertures, can promote and/or facilitate formation of surface protuberances on the graft material as portions of the same are drawn toward the apertures while under vacuum. In one aspect, an amount of ECM material is retained in such a mold, and needles or other material-displacing objects are inserted through some or all of the mold apertures and a distance into the ECM material, thereby displacing volumes of the ECM material. This can be performed when the graft material is hydrated, partially hydrated or dehydrated. In some forms, with needles inserted in a hydrated ECM material and providing passages therein, the material is subjected to conditions (e.g., freezing and/or dehydrating conditions) which, alone or in combination with one or more other conditions, cause or allow the passages to be generally retained in the ECM material after the needles are removed.

In one embodiment, one or more sheets of hydrated ECM material are suitably wrapped and/or randomly packed around a mandrel, and then a mold having a plurality of holes extending through a wall of the mold is placed around the material-covered mandrel, for example, so that an amount of pressure is placed on the ECM material. The mandrel can then optionally be removed. Thereafter, needles or other material-displacing objects are inserted through some or all of the holes and at least partially through the ECM material, thereby displacing volumes of the ECM material. The ECM material is then at least partially dried. In some aspects, a suitable lyophilization technique is employed, e.g., one with or without a pre-freezing step as described herein. In these or other drying methods in which needles or other penetrating elements are to be left within the mass during drying, these elements can optionally be provided with a plurality of apertures or holes or can otherwise be sufficiently porous to facilitate the drying operation by allowing the passage of hydrate from the wet mass. In one embodiment, a hydrated ECM material with emplaced needles can be subjected to freezing conditions so that the material and any contained hydrate become substantially frozen. Thereafter, the needles can be removed from the ECM material, and the remaining construct (with the frozen material passages substantially retaining their shape) can be placed under a vacuum so that the frozen hydrant sublimes from the material, thereby resulting in a dry graft construct with retained passages therein.

In other modes of operation, passage-forming structures can be incorporated integrally into a mold so that passageways are formed upon introducing the starting material in and/or on the mold. In these aspects, the passage-forming structures can be part of the mold (e.g., extend from a surface of the mold), or they can be separate objects attached or otherwise coupled to the mold, to provide the desired passage or passages through the ultimately-formed graft body.

Although not necessary to broader aspects of the invention, in some aspects, the formation of such a graft construct comprises wrapping one or more sheets of hydrated graft material around a mandrel a number of times. The resulting roll of graft material is then introduced into a mold, and the mandrel is removed (optional), e.g., before or after applying the mold. Thereafter, multiple material-displacing objects such as but not limited to needles are forced through apertures in the mold and into the hydrated graft material, and the material is subjected to one or more drying techniques such as a lyophilization process. In other aspects, the formation of such a graft construct includes placing a flowable graft material into a mold and then subjecting the graft material to further processing. For example, a flowable ECM material mass, such as a gel, paste or putty, potentially incorporating a particulate ECM material, can be placed into a mold, and then with volumes of material displaced in the mass (e.g., by penetrating needles), the ECM material can be dried or otherwise caused to form an integral piece to provide a graft body having passages therein. Illustratively, each of the passages can be provided by forcing a single object through the material mass, or alternatively, where a mandrel is left in place to form a longitudinal lumen, by forcing two objects into the mass and toward one another from opposite directions until they abut the mandrel. The mass can then be processed to a solid graft body as discussed herein.

Some of the materials used in the present invention have a level or degree of porosity. In certain embodiments, these materials' resistance to hydration is altered by manipulating their bulk density and/or level of porosity. Illustratively, the porosity of an ECM material can be lowered by drying the material under compression. In general, compressing a pliable, open matrix material, such as a pliable ECM material, increases the material's bulk density and decreases the material's porosity by decreasing the size of the voids in the open matrix. As is the case in certain aspects of the invention, when such a material is dried while being compressed, particularly under vacuum pressing conditions, the open matrix structure can become generally fixed in this relatively higher bulk density, lower porosity state (i.e., in a relatively more collapsed state), thereby providing a stiffer, and potentially more hydration resistant, material. It should be noted that different compressing and drying methods, including different degrees of compressing and drying, can be designed through routine experimentation so as to allow for a material having an optimal degree of material bulk density and/or porosity for a particular application. As well, other suitable technique for altering a material's bulk density and/or porosity can be in the present invention including but not limited subjecting a crosslinkable material to a suitable crosslinking technique.

In certain embodiments, material in a core region of a device provides a hydration resistant component. This core material can include, for example, material that has been somehow treated to increase its resistance to hydration. Additionally or alternatively, a core material can include one or more core members (e.g., formed constructs, material pieces, etc.) that are at least partially surrounded by other parts of the device. Material occurring in a core region of a device can include material that is rigid, malleable, semi-flexible, or flexible. Also, a device core may be separable from other parts of the device, or alternatively, it may be essentially inseparable. Although not necessary to broader aspects of the invention, in one form, a device provides a designated opening (e.g., a lumen or other passage) into which a core material can be removably positioned. As well, a device core can exhibit a variety of shapes, and may be formed with one or more of a variety of materials including some that are naturally derived and some that are non-naturally derived. In some forms, a device core and at least one other part of the device are formed with a sheet-form material. In other forms, core material and/or non-core material of a device are formed with non sheet-form material.

In one embodiment, a device core and another part of the device while dissimilar in their resistance to hydration are comprised of one or more of the same materials. Illustratively, a fistula plug for delivery into a fistula tract can include a plug body and a core material received in the plug body, wherein the plug body and core material are each comprised of a collagen-containing material, yet the core material is adapted to be less receptive to hydration than the plug body. In another embodiment, a fistula plug includes a plug body and a core material received in the plug body, wherein the plug body is comprised of a dried, remodelable collagenous material, and the core material is comprised of a resorbable synthetic material that is somewhat more resistant to hydration than the dried, remodelable collagenous material. Such a plug can be formed, for example, by wrapping one or more layers of hydrated, remodelable collagenous material around the resorbable core material one or more times, and then subjecting the plug to drying conditions (optionally while compressing the remodelable collagenous material around the core material).

Referring now to FIG. 2, shown is another fistula plug 60 of the present invention. Plug 60 includes a plug body 61 comprised of a rolled sheet-form material. Plug 60 also includes a core material 62, which is surrounded by this rolled sheet-form material. Plug body 61 is formed with a first hydratable material. Core material 62 is formed with a second hydratable material having less receptivity to hydration than the first hydratable material. A plug such as plug 60 can be formed in any suitable manner. For instance, plug body 61 can be formed directly around core material 62. In some forms, plug body 61 is formed separately (e.g., around a mandrel similar in diameter to core material 62) such that a passage is formed in the plug body 61. Thereafter, core material 62 is positioned in this passage. In this particular embodiment, plug body is formed with a single layer of material. In alternative embodiments, plug body is formed with two or more layers of material, wherein a given layer can have the same or different receptivity to hydration than another layer. For example, plug body 31 depicted in FIG. 1A could be adapted to receive a core material such as core material 62. In one illustrative embodiment, core material 62 and plug body 61 are both formed with a sheet-form collagen-containing material except that the core material is adapted to be less receptive to hydration than the plug body. Illustratively, a core material including one or more pieces of a sheet-form collagenous material can be subjected to particular drying conditions and/or other treatments to enhance its resistance to hydration relative to a plug body which also includes one or more pieces of a sheet-form collagenous material but that is subjected to different treatments or no treatments. In one form, a core material includes multiple pieces of a hydrated sheet-form collagenous material that are arranged into a particular configuration by folding, rolling and/or twisting the pieces together and then allowed to air dry in this configuration. Once at least partially dried, this core material provides a relatively more rigid member around which a sheet-form plug body can be positioned.

In some embodiments, a substance coating a surface of one or more portions of a device provides a hydration resistant component. In one embodiment, an inventive fistula plug includes a plug body comprised of a hydratable material, as well as a hydration resistant coating material coating a surface of the plug body. Such a coating material can be used to coat all or part of an existing plug device that is otherwise formed and ready for use. Alternatively, at least part of a plug body can be coated as the plug device is being formed. In this regard, a coating material can coat what is considered an interior surface of a plug body and/or an exterior surface of a plug body. Illustratively, a surface of a material layer that is used in the formation of an inventive device can be coated before it is used to form all or part of a plug body. Thus, any part of an inventive device such as those shown in FIGS. 1 and 2 can be coated with a hydration resistant coating material.

With reference now to FIG. 3, shown is a fistula plug 90 according to another embodiment of the present invention. Plug 90 includes a plug body 91 comprised of three elongate material segments braided together. The three material segments are each comprised of a hydratable material coated with a hydration resistant coating material. Plug 90 also includes a “leading” distal portion 92, and a capping member 93, both of which are optionally included. Such a leading distal portion, when incorporated into an inventive plug, can exhibit a variety of shapes and sizes, and in some forms, will be particularly configured to enhance the travel of the plug into and through a fistula tract. For example, a suitable distal portion can include a tapered portion and/or have a dome-shaped or otherwise rounded tip, which can help avoid substantially cutting or tearing soft tissues in and around a fistula tract. A band 95 is positioned around plug body 91, and is effective to at least help maintain the three segments in a braided configuration.

Distal portion 92 can be formed with one or more of a variety of materials including some that are naturally derived and some that are non-naturally derived. When an inventive device is equipped with a distal portion such as distal portion 92, this portion and any other part of the device (e.g., a plug body such as plug body 91) may be formed as a single unit (e.g., from an amount of the same material), or alternatively, such device parts may be formed separately and then combined with one another, for example, using an adhesive, by suturing, using mechanical fastener(s), and/or any other suitable joining means. Other effective ways to assemble two or more device components will be recognized by those skilled in the art, and therefore, are encompassed by the present invention. When formed separately, any two device components may or may not be comprised of the same biocompatible material(s).

In embodiments where two or more parts of a device (e.g., a distal portion such as distal portion 92 and a plug body such as plug body 91) are formed as separate constructs, the two may be coupled to one another with an absorbable coupling element. Such coupling elements can exhibit a variety of configurations, and in some aspects, take the form of an adhesive or one or more hooks, fasteners, barbs, straps, suture strands, or suitable combinations or variations thereof. Coupling elements of this sort may be comprised of one or more of a variety of suitable biocompatible materials exhibiting a rate of degradation upon implantation in vivo, such as but not limited to a 2-0 vicryl suture material. Illustratively, a coupling element can be adapted to desirably hold a distal portion and plug body in association with another during product handling and implantation, and then upon implantation, to degrade at a desirable rate.

In some modes of operation, plug 90 is positioned in a fistula tract by passing distal end portion 92 through a secondary opening and toward a primary fistula opening in the alimentary canal. Plug 90 can be advanced until distal portion 92 is positioned in the primary opening and extends a distance into the alimentary canal. Sometime after implantation, the distal portion and plug body, at least due in part to degradation of the coupling element, can uncouple or otherwise disengage from one another, allowing the distal portion to be discarded, e.g., to pass through and out of the bowel with naturally occurring fecal mater. In some instances, this decoupling can be facilitated and/or promoted by naturally occurring forces generated during peristalsis.

When present in a device, a capping member such as capping member 93 can exhibit a variety of shapes and sizes, and may be formed with one or more of a variety of materials including some that naturally derived and some that are non-naturally derived. Illustratively, a capping member can include one or more objects (e.g., devices, pieces of material, etc.) that, together or alone, exhibit a three-dimensional rectilinear or curvilinear shape. Suitable three-dimensional rectilinear shapes can have any suitable number of sides, and can include, for example, cubes, cuboids, tetrahedrons, prisms, pyramids, wedges, and variations thereof. Suitable three-dimensional curvilinear bodies can include, for example, spheres, spheroids, ellipsoids, cylinders, cones, and any suitable variations thereof (e.g., a segment of a sphere, or a truncated cone, etc.).

Illustratively, capping members useful in the invention can be prepared and utilized, for example, as described in International Patent Application Serial No. PCT/US2006/024260, filed Jun. 21, 2006, and entitled “IMPLANTABLE GRAFT TO CLOSE A FISTULA” (Cook Biotech Incorporated); and U.S. Provisional Patent Application Ser. No. 60/763,521, filed Jan. 31, 2006, and entitled “FISTULA GRAFTS AND RELATED METHODS AND SYSTEMS FOR TREATING FISTULAE” (Cook Biotech Incorporated), which are hereby incorporated by reference in their entirety.

In accordance with the present invention, a plug can incorporate a variety of other adaptations to enhance its travel through a body passageway or other opening. In some embodiments, a plug body, or a portion thereof, is particularly configured to enhance its ability to articulate when traveling through the body. Illustratively and referring now to FIG. 4, shown is a partial view of a device that is similar to that shown in FIG. 3 except that it includes a plurality of cuts 100 in the material segments of the plug body. These sorts of articulation adaptations can enhance the travel of a plug body such as plug body 91 through a fistula tract, particularly when negotiation around sharp bends is required. Suitable articulation adaptations can include one or more indentations, scores, thinner portions, etc. in the plug body. These and other adaptations for enhancing articulation of a plug body will be recognized by the skilled artisan, and therefore, are encompassed by the present invention.

The invention provides a variety of other devices having the ability to articulate in some fashion along all or part of the device. In some forms, an inventive device includes two or more plug body segments that are directly or indirectly joined to one another in the device in such a way that device exhibits some degree of lateral flexibility. These segments may be joined to one another in any suitable manner. Illustratively, an inventive plug can include an articulating plug component comprised of two or more elongate plug body segments hingedly connected to one another in succession. In one form, such a plug further comprises a covering material positioned around the two or more elongate plug body segments, for example, a sheet-form material wrapped around at least part of the two or more elongate plug body segments. The two or more elongate plug body segments can each exhibit a variety of shapes, sizes and configurations, and the segments may be hingedly connected to one another in any suitable manner, e.g., with suture material, one or multiple-part coupling devices, and/or other objects that are effective to hold or at least help hold the segments together, etc. Illustratively, suitable plug body segments can include some that are formed with rolled and/or folded sheet-form material, braided strips of material, etc.

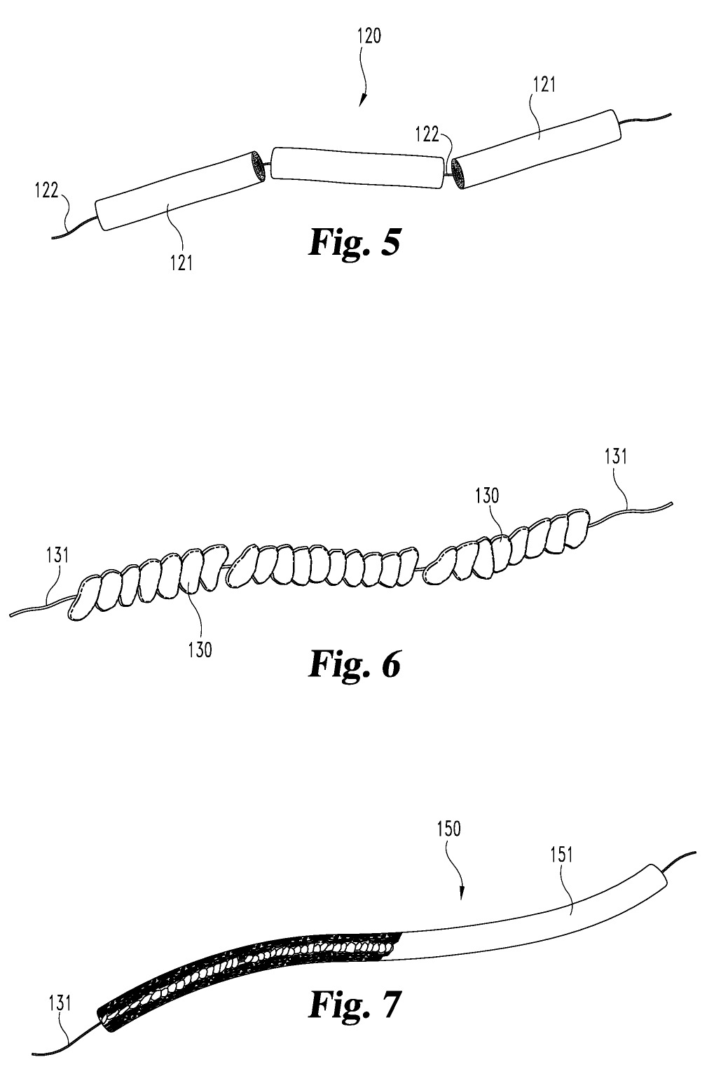

With reference now to FIG. 5, shown is another fistula plug 120 of the present invention. Plug 120 includes three elongate plug body segments 121, which are each comprised of a rolled sheet-form material exhibiting a generally cylindrical shape. A suture strand 122 extends through each of the plug body segments 121, and is effective to unite the three plug body segments in succession. Although not necessary to broader aspects of the invention, in this particular embodiment, all or a portion of the outer surface of each of the plug body segments 121 is coated with a hydration resistant coating material.

In other embodiments, one or more plug body segments to be included in such a device are formed similarly to those plug bodies described elsewhere herein (e.g., as depicted in FIGS. 1-4). Illustratively, formation of a plug body segment can involve rolling, folding or otherwise overlaying one or more pieces of material in a random or non-random fashion. For example, a plug body segment can comprise a spirally wound piece of material such as that shown in FIG. 6, where plug body segments 130 are formed by spirally winding a piece of material around a suture 131. In some embodiments, the material used is sufficiently malleable to enable a segment to maintain its spiral configuration once formed. In other embodiments, the ends of each spirally wound piece of material are substantially fixed in place to enable the segment to maintain its spiral configuration, for example, by drying and/or otherwise treating the material (e.g., vacuum pressing), by somehow tucking the ends into another portion of the segment, securing the ends to the suture and/or another portion of the plug body segment, etc. Other ways of maintaining a desirable plug segment configuration will be recognized by those skilled in the art, and therefore, are encompassed by the present invention.

FIG. 7 shows another fistula plug 150 of the present invention, which is similar to that shown in FIG. 6 except that it additionally includes a cover material 151 covering elongate plug body segments 130. Such a covering material can be configured in a variety of manners, and may be formed with one or more of a variety of materials including some that are naturally derived and some that are non-naturally derived. In the current embodiment, cover material 151 is comprised of a sheet-form material rolled around plug body segments 130 to exhibit a generally cylindrical form. Additionally or alternatively, a suitable cover material may be comprised of a “non sheet-form” material, for example, material whose formation involves extrusion, using a mold or form, construction around a mandrel, and/or combinations or variations thereof. These cover materials may be formed directly around one or more plug body segments, or alternatively, formed separately from a plug body segment and then later combined with a plug body segment. In some forms, a flowable material is sprayed onto or otherwise applied to a plug body segment as part of forming a suitable cover material. While shown in the current device, suture 131 is optional.

Continuing with FIG. 7, plug body segments 130 include material that is more resistant to hydration than material contained in cover material 151, although such is not necessary to broader aspects of the invention. In another embodiment, plug body segments 130 include material that is less resistant to hydration than material contained in cover material 151. In some aspects, a cover material such as cover material 151 is formed similarly to the plug body depicted in FIG. 1 or is otherwise formed in accordance with the present invention, for example, including a reconstituted or otherwise reassembled collagen-containing material.

Some embodiments of the present invention involve grafts comprised of a hydrated material, wherein the hydrate in the material is frozen. Such grafts find wide use in the medical arts, particularly in treatments that involve placing the grafts on or in the body to replace, repair, augment, and/or otherwise suitably treat wounded, diseased or otherwise damaged or defective bodily tissue. Illustratively, an inventive plug of this sort can be delivered into a body passageway to plug that passageway. In some forms, such frozen, hydrated material comprises a remodelable angiogenic material, for example, an extracellular matrix material such as but not limited to porcine small intestine submucosa.

Freezing the hydrate in a graft material can provide a number of enhancements to the graft, which will be recognized by those skilled in the art. In some instances, one or more handling and/or delivery characteristics of a plug will be enhanced by freezing hydrate in the plug (e.g., with CO₂). Illustratively, providing a plug with a frozen component can enhance the plugs ability to be pushed through a passageway or other opening in the body. When an outer surface of such a plug is frozen, at least a portion of this surface may be warmed and/or lubricated to inhibit the plug from adhering to tissue along the passageway. Additionally, such a plug can be warmed at one or more locations therealong to provide more flexibility to the plug, if desirable. In some instances, freezing hydrate in an elongate plug will increase the column strength of the plug, compared to the same plug in an unfrozen or less frozen state. Frozen hydrate in a plug can also impart a hydration resistant component to a plug. In some instances, selected portions of a hydrated plug body are frozen, for example, in a particular pattern along the body, to provide a plug body having frozen parts and unfrozen or less frozen parts. The frozen parts can enhance the plug's ability to be pushed through a bodily passage, while the unfrozen or less frozen parts can impart a degree of flexibility to the plug, for example, enhancing the plug's ability to articulate when traveling through the passage, compared to a uniformly frozen plug.

An inventive device, or any component thereof, can itself be considered lubricious by those skilled in the art. In some forms, a device or one or more device components will include a layer (e.g., a coating) to enhance the lubricious properties of the component(s). Such a layer may be applied (e.g., by spraying, dip coating, over-extruding or by any other suitable means) to the component(s), and may be comprised of a hydrophilic material such as but not limited to parylene or PTFE. In certain aspects, UV (ultra-violet light)-curable, radiation-curable, photoreactive, photoimmobilizing, and other similar coatings are used. These coatings have in common at least one photoreactive species. Coatings can be made from these species, and then all or a portion of a tissue augmentation device can be coated and the coating cured. Lubricous coating materials include those commercially available from SurModics, Inc., Eden Prairie, Minn., under the trade mark “PhotoLink™.”

Devices of the invention may be used to plug or otherwise fill a variety of passages or other open spaces in the body. In some instances, these open spaces will occur naturally in the body, for example, as a native lumen or other open space in a bodily system, e.g., in an organ or other component of the circulatory, respiratory, digestive, urinary and reproductive, sensory, or endocrine systems. In certain aspects, a space to be filled is one that exists naturally in the body but relates to a disease, defect, deformation, etc. Alternatively, an opening or passage to be filled may be one resulting from an intentional or unintentional trauma to the body including but not limited to some relating to vehicular accidents, gunshots and other similar wounds, etc., as well as some formed by passage of a medical instrument (e.g., a needle, trocar, etc.) through cutaneous, subcutaneous, and/or intracutaneous tissue.

Illustratively, inventive devices, alone or in conjunction with one or more other suitable objects, can be used to occlude, or at least promote and/or facilitate occlusion of, a lumen or other open space in the vasculature, e.g., a blood vessel such as a vein or artery, or a lumen or open space of a fallopian tube, e.g. in a procedure to provide sterility to a female patient. In certain aspects, one or more assemblies of the invention are deployed within the venous system (e.g., within the greater and/or lesser saphenous vein) to treat complications, such as a varicose vein conditions. In other embodiments, inventive assemblies are used as contraceptive devices. In preferred embodiments, assemblies of the invention can be used to plug or otherwise fill fistulae such as but not limited to urethro-vaginal fistulae, vesico-vaginal fistulae, tracheo-esophageal fistulae, gastro-cutaneous fistulae, and any number of anorectal fistulae, such as recto-vaginal fistula, recto-vesical fistulae, recto-urethral fistulae, or recto-prostatic fistulae.

In accordance with the present invention, a device can be positioned at a treatment site in any suitable manner including some that involve directly or indirectly pushing and/or pulling the device in the body. As well, such positioning can be performed directly by hand in situations where such access is possible, although in some embodiments, positioning the device will additionally or alternatively involve the use of one or more instruments. In one aspect, a pulling device (e.g., a suture, grasping tool, etc.), which is attached to or otherwise associated with the device, is used to at least help position the device in a desirable location.

Inventive devices, in certain forms, can include a variety of synthetic polymeric materials including but not limited to bioresorbable and/or non-bioresorbable plastics. Bioresorbable, or bioabsorbable polymers that may be used include, but are not limited to, poly(L-lactic acid), polycaprolactone, poly(lactide-co-glycolide), poly(hydroxybutyrate), poly(hydroxybutyrate-co-valerate), polydioxanone, polyorthoester, polyanhydride, poly(glycolic acid), poly(D,L-lactic acid), poly(glycolic acid-co-trimethylene carbonate), polyhydroxyalkanaates, polyphosphoester, polyphosphoester urethane, poly(amino acids), cyanoacrylates, poly(trimethylene carbonate), poly(iminocarbonate), copoly(ether-esters) (e.g., PEO/PLA), polyalkylene oxalates, and polyphosphazenes. These or other bioresorbable materials may be used, for example, where only a temporary blocking or closure function is desired, and/or in combination with non-bioresorbable materials where only a temporary participation by the bioresorbable material is desired.

Non-bioresorbable, or biostable polymers that may be used include, but are not limited to, polytetrafluoroethylene (PTFE) (including expanded PTFE), polyethylene terephthalate (PET), polyurethanes, silicones, and polyesters and other polymers such as, but not limited to, polyolefins, polyisobutylene and ethylene-alphaolefin copolymers; acrylic polymers and copolymers, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins, polycarbonates; polyoxymethylenes; polyimides; polyethers; epoxy resins, polyurethanes; rayon; and rayon-triacetate.

In certain embodiments, an inventive device includes a radiopaque element. For example, a device can include a radiopaque substance or device such as but not limited to a radiopaque coating, attached radiopaque object, or integrated radiopaque substance useful for determining the location of the device, or a component thereof, in the body. In certain forms, a device component such as distal portion 92 can be formed of a polymeric material loaded with a particulate radiopaque material. In this regard, any suitable radiopaque substance, including but not limited to, tantalum such as tantalum powder, can be incorporated into an inventive component. Other radiopaque markers may be comprised of gold, bismuth, iodine, and barium, as well as other suitable radiopaque materials.

In certain aspects of the invention, treatment of a fistula includes an endoscopic visualization (fistuloscopy) step that is performed prior to implanting a fistula plug. Such endoscopic visualization can be used, for example, to determine the shape and size of a fistula, which in turn can be used to select an appropriately sized and shaped fistula graft device for treating the fistula. Illustratively, a very thin flexible endoscope can be inserted into a secondary opening of the fistula and advanced under direct vision through the fistula tract and out through the primary opening. By performing fistuloscopy of the fistula, the primary opening can be accurately identified. Also, certain fistula treatment methods of the invention include a fistula cleaning step that is performed prior to implanting a fistula graft. For example, an irrigating fluid can be used to remove any inflammatory or necrotic tissue located within the fistula prior to engrafting the graft device. In certain embodiments, one or more antibiotics are applied to the fistula graft device and/or the soft tissues surrounding the fistula as an extra precaution or means of treating any residual infection within the fistula.

Additionally, an inventive device, or any component thereof, can incorporate an effective amount of one or more antimicrobial agents and/or therapeutic agents otherwise useful to inhibit the population of the device and surrounding tissue with bacteria and/or other deleterious microorganisms. Illustratively, a device can be coated with one or more antibiotics such as penicillin, tetracycline, chloramphenicol, minocycline, doxycycline, vancomycin, bacitracin, kanamycin, neomycin, gentamycin, erythromycin and cephalosporins. Examples of cephalosporins include cephalothin, cephapirin, cefazolin, cephalexin, cephradine, cefadroxil, cefamandole, cefoxitin, cefaclor, cefuroxime, cefonicid, ceforanide, cefotaxime, moxalactam, ceftizoxime, ceftriaxone, and cefoperazone, and antiseptics (substances that prevent or arrest the growth or action of microorganisms, generally in a nonspecific fashion) such as silver sulfadiazine, chlorhexidine, glutaraldehyde, peracetic acid, sodium hypochlorite, phenols, phenolic compounds, iodophor compounds, quaternary ammonium compounds, and chlorine compounds. These or other therapeutic agents can be incorporated directly on or in an inventive device, or they can be incorporated with a suitable binder or carrier material, including for instance hydrogel materials. The carrier or binder coating can be applied to the device by any suitable means including, for example, spraying, dipping, etc. as known in the art. The antimicrobial or other therapeutic agent can be added to the carrier/binder coating either prior to or after application of the coating to the device.

Further, inventive fistula plug devices can be adapted for delivery into one or multiple fistula tracts in a given medical procedure. In this context, the term “fistula tract” is meant to include, but is not limited to, a void in soft tissues extending from a primary fistula opening, whether blind-ending or leading to one or more secondary fistula openings, for example, to include what are generally described as simple and complex fistulae. In cases of complex fistulae, for example a horse-shoe fistula, there may be one primary opening and two or more fistula tracts extending from that opening. In such instances, a fistula graft may be delivered to any of the fistula tracts.

All publications and patent applications cited in this specification are herein incorporated by reference as if each individual publication or patent application were specifically and individually indicated to be incorporated by reference. Further, any theory, mechanism of operation, proof, or finding stated herein is meant to further enhance understanding of the present invention, and is not intended to limit the present invention in any way to such theory, mechanism of operation, proof, or finding. While the invention has been illustrated and described in detail in the drawings and foregoing description, the same is to be considered as illustrative and not restrictive in character, it being understood that only selected embodiments have been shown and described and that all equivalents, changes, and modifications that come within the spirit of the inventions as defined herein or by the following claims are desired to be protected.

What is claimed is:

1. A fistula plug for delivery into a fistula tract, comprising:
a plug body comprised of a dried collagen-containing material; and
a core material received in the plug body, wherein the core material is less receptive to hydration than the plug body.

2. The fistula plug of claim 1, wherein the dried collagen-containing material comprises a remodelable extracellular matrix material.

3. The fistula plug of claim 1, wherein the core material is comprised of a naturally derived biocompatible material.

4. The fistula plug of claim 1, wherein the core material is comprised of a collagen-containing material.

5. The fistula plug of claim 1, wherein the core material is comprised of a non-naturally derived material.

6. The fistula plug of claim 1, wherein the core material is comprised of a synthetic polymeric material.

7. The fistula plug of claim 1, wherein the core material is removably received in the plug body.

8. The fistula plug of claim 7, wherein the plug body has a lumen defined therein, and wherein the core material is removably received in the plug body lumen.

9. A fistula plug for delivery into a fistula tract, comprising:
a plug body comprised of a hydratable material; and
a hydration resistant material component incorporated on or in the plug body.

10. The fistula plug of claim 9, wherein the hydratable material comprises a remodelable material.

11. The fistula plug of claim 9, wherein the hydratable material comprises a collagen-containing material.

12. The fistula plug of claim 9, wherein the hydratable material comprises an extracellular matrix material.

13. The fistula plug of claim 12, wherein the extracellular matrix material comprises submucosa.

14. The fistula plug of claim 12, wherein the extracellular matrix material comprises serosa, pericardium, dura mater, peritoneum, or dermal collagen.

15. The fistula plug of claim 9, wherein the hydratable material comprises a synthetic polymeric material.

16. The fistula plug of claim 9, wherein the hydration resistant material component comprises a sheet-form material incorporated on or in the plug body.

17. The fistula plug of claim 9, wherein the hydration resistant material component comprises a non-sheet-form material incorporated on or in the plug body.

18. The fistula plug of claim 9, wherein the hydratable material is comprised of a porous material having a plurality of interconnected spaces therein, and wherein the hydration resistant material component includes material residing in the interconnected spaces.

19. The fistula plug of claim 9, wherein the hydration resistant material component adds column strength to the plug body.

20. A method of treating a fistula having at least a primary fistula opening, a secondary fistula opening, and a fistula tract extending therebetween, the method comprising:
delivering into the fistula tract a fistula plug comprising:
a plug body comprised of a hydratable material; and
a hydration resistant material component incorporated on or in the plug body.

21. A fistula plug for delivery into a fistula tract, comprising:
a plug body comprised of a rolled sheet-form material, the plug body including a collagen-containing material layer and a hydration resistant material layer.

22. The fistula plug of claim 21, wherein the collagen-containing material layer surrounds at least a portion of the hydration resistant material layer.

23. The fistula plug of claim 21, wherein the hydration resistant material layer surrounds at least a portion of the collagen-containing material layer.

24. The fistula plug of claim 23, wherein a second collagen-containing material layer surrounds at least a portion of the hydration resistant material layer.

25. The fistula plug of claim 21, wherein the plug body is comprised of a rolled multilaminate sheet-form material.

26. The fistula plug of claim 21, wherein the plug body has a generally cylindrical portion.

27. The fistula plug of claim 21, wherein the plug body has a generally conical portion.

28. The fistula plug of claim 21, wherein the rolled sheet-form material includes material layers compressed and bonded so as to form a substantially unitary construct.

29. A fistula plug for delivery into a fistula tract, comprising:
a plug body comprised of a collagen-containing material; and
a hydration resistant coating material coating a surface of the plug body.

30. The fistula plug of claim 29, wherein the coating material coats an exterior surface of the plug body.

31. The fistula plug of claim 29, wherein the coating material coats an interior surface of the plug body.

32. The fistula plug of claim 29, wherein the collagen-containing material is comprised of a material layer, and wherein the coating material coats a surface of the material layer.

33. A fistula plug for delivery into a fistula tract, comprising:
an articulating plug component comprised of two or more elongate plug body segments hingedly connected to one another in succession.

34. The fistula plug of claim 33, further comprising a covering material positioned around the two or more elongate plug body segments.

35. The fistula plug of claim 34, wherein the covering material comprises a sheet-form material wrapped around the two or more elongate plug body segments.

36. The fistula plug of claim 34, wherein the covering material comprises a non-sheet-form material.

37. The fistula plug of claim 33, wherein the two or more elongate plug body segments are hingedly connected to one another with a suture material.

38. The fistula plug of claim 33, wherein at least one of the two or more elongate plug body segments comprise a rolled sheet-form material.

39. The fistula plug of claim 33, wherein at least one of the two or more elongate plug body segments comprise a braided material.

40. A method plugging a passageway in the body, comprising:
delivering into the body passageway a plugging device comprised of a hydrated remodelable angiogenic material, wherein the hydrate in the material is frozen.
